(19) United States
(12) Patent Application Publication
(10) Pub. No.: US 2008/0260696 A1
(43) Pub. Date: Oct. 23, 2008

(54) Method for decreasing inflammation and oxidative stress in mammals

(76) Inventors: Stefan Patrick Massimino, Portland, OR (US); Gary Mitchell Davenport, Dayton, OH (US); Michael Griffin Hayek, Dayton, OH (US); George Roth, Pylesville, MD (US); Donald K. Ingram, Ellicott City, MD (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/012,318

(22) Filed: Feb. 1, 2008

Related U.S. Application Data
(60) Provisional application No. 60/898,854, filed on Feb. 1, 2007.

(51) Int. Cl. A61K 35/74; A61K 31/7004; A61K 36/00

(52) U.S. Cl. 424/93.4; 514/23; 424/777; 424/725

(57) ABSTRACT

The present invention is directed to a method for decreasing inflammation and oxidative stress in a mammal comprising; administration to a mammal a composition comprising a glucose anti-metabolite; and wherein said composition comprises amounts of the glucose anti-metabolite sufficient to decrease a level of an oxidized glutathione and/or increase the ration of reduced glutathione to oxidized glutathione in the blood of the mammal subsequent to administration of the glucose anti-metabolite.

This application claims the benefit of U.S. Provisional Application No. 60/898,854, filed Feb. 1, 2007.

### FIELD OF THE INVENTION

The present invention is directed to a method for decreasing inflammation and oxidative stress in a mammal comprising; administration to a mammal a composition comprising a glucose anti-metabolite; and wherein said composition comprises amounts of the glucose anti-metabolite sufficient to decrease a level of an oxidized glutathione and/or increase the ratio of reduced glutathione to oxidized glutathione in the blood of the mammal subsequent to administration of the glucose anti-metabolite.

### BACKGROUND OF THE INVENTION

The state of oxidative stress occurs when there is an imbalance between prooxidant and antioxidant mechanisms. An overabundance of prooxidants can produce molecular and cellular damage. Increased oxidative stress is associated with various diseases such as coronary heart disease, neurodegenerative diseases, arthritis, and cataract formation, as well as immune system dysregulation. Antioxidant mechanisms exist in an animal such as antioxidant enzymes and other small molecular antioxidants that can protect against harmful effects of free radicals. The level of oxidative stress may be associated with a disease, and used to identify an animal at risk for the development of the disease, or monitor therapies directed to the disease.

Reduced glutathione (GSH) is a linear tripeptide of L-glutamine, L-cysteine, and glycine. Technically, N-L-gamma-glutamyl-cysteinyl glycine or L-glutathione, the molecule has a sulfhydryl (SH) group on the cysteinyl portion, which accounts for its strong electron-donating character. Glutathione (GSH) is a major antioxidant in animal tissues. Under the effect of glutathione peroxidase, GSH can remove H.sub.2 O.sub.2 at a high rate and in the process itself becomes oxidized glutathione (GSSG). Oxidized glutathione (GSSG) is known as a dimmer of tripeptide glutathione (gamma.-glutamyl-cysteinyl-glycine). The GSSG must be converted back to GSH by the enzyme glutathione reductase.

Glutathione is recognized as a potent antioxidant and enzyme cofactor and plays a critical role in regulating cellular activity. Free radical and other oxidative agents can deplete GSH. The homeostatic glutathione redox cycle attempts to maintain GSH levels as glutathione is being consumed. Amounts of glutathione available from foods are limited, and oxidative depletion can outpace synthesis. GSH is an extremely important cell protectant. GSH directly quenches reactive hydroxyl free radicals, other oxygen-centered free radicals, and radical centers on DNA and other biomolecules. GSH protects skin, lens, cornea, and retina against radiation damage, and the biochemical foundation of P450 detoxication in the liver, kidneys, lungs, intestinal epithelia, and other organs. Oxidative stressors that can deplete GSH include aging, ultraviolet and other radiation; viral infections; environmental toxins, household chemicals, and heavy metals; surgery, inflammation, burns, septic shock; and dietary deficiencies of GSH precursors and enzyme cofactors.

GSH is under tight homeostatic control both intracellularly and extracellularly. A dynamic balance is maintained between GSH synthesis, GSH recycling from GSSG/oxidized glutathione, and its utilization. The balance between oxidative stress and the defensive systems of the cells and organs have crucial importance. It has been found that glucose anti-metabolite, avocados, avocado extract and mannoheptulose have potent activity in the maintenance of the level of reduced glutathione, reducing the level of oxidized glutathione, and increasing the ratio of reduced to oxidized glutathione (GSH/GSSG).

When cells are exposed to increased oxidative stress, the ratio of GSH/GSSG will decrease, as a consequence of GSSG accumulation. Therefore, the measurement of the GSH/GSSG ratio provides a significant index to evaluate the state of oxidative stress in a mammal.

There still exists a need for a method for decreasing inflammation and oxidative stress in a mammal including humans and companion animals through the maintenance of the GSH level and decrease in GSSG levels in the blood of a mammal which results in a healthier mammal, enhancement of quality of life of a mammal, and increase the length of the lifespan of a mammal.

It is therefore an object of the present invention to provide a method for decreasing inflammation and oxidative stress in a mammal comprising; administration to a mammal a composition comprising a glucose anti-metabolite, avocado, avocado extract or mannoheptulose; and wherein said composition comprises amounts of the glucose anti-metabolite, avocado, avocado extract or mannoheptulose sufficient to decrease a level of oxidized glutathione and/ or increase the ratio of reduced glutathione to oxidized glutathione in the blood of the mammal subsequent to administration of the glucose anti-metabolite, avocado, avocado extract or mannoheptulose.

### SUMMARY OF THE INVENTION

The present invention is directed to a method for decreasing inflammation and oxidative stress in a mammal comprising; administration to a mammal a composition comprising mannoheptulose; and wherein said composition comprises amounts of the mannoheptulose sufficient to increase a ratio of reduced glutathione to oxidized glutathione in the blood of the mammal subsequent to administration of the mannoheptulose.

The present invention further relates to a method for decreasing inflammation and oxidative stress in a mammal comprising; administration to a mammal a composition comprising a glucose anti-metabolite; and wherein said composition comprises amounts of the glucose anti-metabolite sufficient to increase a ratio of reduced glutathione to oxidized glutathione in the blood of the mammal subsequent to administration of the glucose anti-metabolite.

The present invention further relates to a method for decreasing inflammation and oxidative stress in a mammal comprising; administration to a mammal a composition comprising avocado; and wherein said composition comprises amounts of the avocado sufficient to increase a ratio of reduced glutathione to oxidized glutathione in the blood of the mammal subsequent to administration of the avocado.

The present invention further relates to a method for decreasing inflammation and oxidative stress in a mammal comprising; administration to a mammal a composition comprising avocado extract; and wherein said composition comprises amounts of the avocado extract sufficient to increase a ratio of reduced glutathione to oxidized glutathione in the blood of the mammal subsequent to administration of the avocado extract.

The present invention further relates to a method for decreasing inflammation and stress in a mammal comprising; administration to a mammal a composition comprising mannoheptulose; and wherein said composition comprises amounts of the mannoheptulose sufficient to decrease a level of an oxidized glutathione in the blood of the mammal subsequent to administration of the mannoheptulose.

The present invention further relates to a method for decreasing inflammation and oxidative stress in a mammal comprising; administration to a mammal a composition comprising avocado extract; and wherein said composition comprises amounts of the avocado extract sufficient to increase a ratio of reduced glutathione to oxidized glutathione in the blood of the mammal subsequent to administration of the avocado extract.

### BRIEF DESCRIPTION OF THE DRAWINGS

FIG. 1 is the total GSHₜ reaction rate for an untreated sample;

FIG. 2 is the reaction rate for a M2VP treated sample and the GSSG Blank;

FIG. 3 is the total GSHₜ calibration curve; and

FIG. 4 is the GSSG calibration curve.

### DETAILED DESCRIPTION OF THE INVENTION

The method for the present invention comprises decreasing inflammation and oxidative stress in a mammal comprising; administration to a mammal a composition comprising a mannoheptulose; and wherein said composition comprises amounts of the mannoheptulose sufficient to increase a ratio of reduced glutathione to oxidized glutathione in the blood of the mammal subsequent to administration of the mannoheptulose.

These and other limitations of the compositions and methods of the present invention, as well as many of the optional ingredients suitable for use herein, are described in detail hereinafter.

As used herein, the term “adapted for use” means that the composition described can meet the American Association of Feed Control Officials (AAFCO) safety requirements for providing animal food products for an animal as may be amended from time to time.

As used herein, the term “companion animal” means an animal preferably including (for example) dogs, cats, kitten, puppy, senior dog, senior cat, adult dog, adult cat, horses, cows, pigs, rabbits, guinea pig, hamster, gerbil, ferret, zoo mammals, fish, birds and the like. Dogs, cats, kitten, puppy, senior dog, senior cat, adult dog, adult cat are particularly preferred.

As used herein, the term “composition” means a composition that can be administered to a human that is orally ingested by the human, bars, pills, capsules, administered to companion animal that is orally ingested by a companion animal, supplements for a companion animal, pet food, dog food, cat food, treats, biscuits, raw hide, treats, chews, fillers, gravy, sauce, beverage, supplemental water, and combinations thereof. The composition can be wet, moist, and/or dry.

The term “complete and nutritionally balanced” as used herein, unless otherwise specified, refers to a composition having all known required nutrients in proper amounts and proportions based upon the recommendation of recognized authorities in the field of companion animal nutrition.

As used herein, the term “endogenous” means originating or produced within a blood or tissue sample.

As used herein, the term “GSH” means endogenous reduced glutathione.

As used herein, the term “total GSHₜ” includes reduced GSH in combination with reduced GSH derived from the conversion of GSSG to two molecules of reduced GSH as determined by the method described herein.

As used herein, the term “GSSG” means oxidized glutathione.

As used herein, the term “mammal” includes humans and/or companion animals.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The composition and methods of the present invention can comprise, consist of, or consist essentially of, the essential elements and limitations of the invention described herein, as well as any additional or optional ingredients, components, or limitations described herein or otherwise useful in compositions intended for mammal consumption.

### Method

The present invention is a method for decreasing inflammation and oxidative stress in a mammal. The method comprises administration to a mammal a composition comprising a glucose anti-metabolite or avocado or mannoheptulose, or avocado extract; and wherein the composition comprises amounts of the glucose anti-metabolite or avocado or mannoheptulose or avocado extract sufficient to decrease a level of oxidized glutathione and/or increase ratio of reduced glutathione to oxidized glutathione in the blood of the mammal subsequent to administration of the glucose anti-metabolite and/or avocado and/or mannoheptulose, and/or avocado extract.

### Composition Form

The compositions are adapted for use by mammals. The composition of the present invention is preferably administered to decrease inflammation and oxidative stress by decreasing the level of an oxidized glutathione and/or increasing the ratio of reduced glutathione to oxidized glutathione in the blood of a mammal. The composition of the present invention can be a moist composition (i.e. those having a total moisture content of from about 16% to 50%, by weight of the product), and/or a wet composition (i.e. those having a total moisture content of greater than 50%, by weight of the product), and/or dry composition (i.e. those having a total moisture content of from about 0% to about 16%, by weight of the product). Unless otherwise described herein, wet composition, moist composition and/ or dry composition are not limited by their composition or method of preparation.

The composition herein can be complete and nutritionally balanced. A complete and nutritionally balanced composition may be compounded to be fed as the sole ration and is capable of maintaining the life and/or promote reproduction without any additional substance being consumed, except for water.

The composition and components of the present invention are preferably for consumption by a mammal, but can also be for consumption by humans. Non-limiting examples of compositions include supplements for an animal, pet food, dog food, cat food, treats, biscuits, raw hide, treats, chews, fillers gravy, sauce, beverage, supplemental water, and combinations thereof.

Additionally, administration in accordance with the present invention may be continuous or intermittent, depending, for example, upon the recipient's physiological condition, whether the purpose of the administration is therapeutic or prophylactic, and other factors known to skilled practitioners.

### Glucose Anti-Metabolite

The method of the present invention comprise administering to a mammal a composition that can comprise a glucose anti-metabolite. The glucose anti-metabolites affects the ratio and level of oxidized glutathione and reduced glutathione present in the blood of a mammal. The decease of the oxidized glutathione and maintenance of the level reduced glutathione decreases inflammation and oxidative stress subsequent to a mammal ingesting a composition comprising glucose anti-metabolites.

The level of an oxidized glutathione (GSSG) in the blood subsequent to administration of a composition comprising a glucose anti-metabolite is from about 0 μM to about 500 μM, from about 5 μM to about 300 μM, from about 5 μM to about 150 μM, from about 10 μM to about 100 μM, as measured by the method described herein.

The level of reduced glutathione (GSH) in the blood subsequent to administration of a composition comprising a glucose anti-metabolite is from about 0 μM to about 4000 μM, from about 1 μM to about 3000 μM, from about 20 μM to about 2500 μM, from about 40 μM to about 2000 μM, as measured by the method described herein.

The level of total glutathione (total GSHₜ) in the blood subsequent to administration of a composition comprising a glucose anti-metabolite is from about 0 μM to about 4000 μM, from about 1 μM to about 3000 μM, from about 20 μM to about 2500 μM, from about 40 μM to about 2000 μM, as measured by the method described herein.

A ratio of reduced glutathione to oxidized glutathione in the blood subsequent to administration of the glucose anti-metabolite is from about 0.1:1 to about 500: 1, from about 0.1:1 to about 250:1, from about 1:1 to about 100:1, from about 1:1 to about 80:1.

Nonlimiting examples of glucose anti-metabolites which are useful herein include 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, and mannoheptulose. Mannoheptulose is preferred for use herein.

The dose of glucose anti-metabolites given to a mammal, on a daily basis, is from about 0.1 mg/kg to about 1000 mg/kg, from about 2 mg/kg to about 100 mg/kg, from about 2 mg/kg to about 10 mg/kg, wherein (as will be commonly understood in the art) the “mg” refers to level of the component and the “kg” refers to kilograms of the mammal or from about 0.0001 gram to about 1 gram of glucose anti-metabolites per kilogram of the mammal. When glucose anti-metabolites is present in a composition, the glucose anti-metabolites is less than about 5%, or less than about 2%, or from about 0.0001% to about 0.5% of the glucose anti-metabolites, all by weight of the composition. The level of component may be determined by one of ordinary skill in the art based on a variety of factors, for example, the form of the pet food composition (e.g., whether a dry composition, moist composition, wet composition, or supplement, or any other form or mixture thereof). The ordinarily skilled artisan will be able to utilize the preferred optimal doses, and use these to determine the optimal level of component within a given pet food composition.

When the glucose anti-metabolite is mannoheptulose the dose of mannoheptulose given to a mammal, on a daily basis, is from about 0.1 mg/kg to about 1 000 mg/kg, from about 1 mg/kg to about 100 mg/kg, from about 2 mg/kg to about 5 mg/kg, wherein (as will be commonly understood in the art) the “mg” refers to level of the mannoheptulose and the “kg” refers to kilograms of the mammal or from about 0.0001 gram to about 1 gram of mannoheptulose per kilogram of the mammal. When mannoheptulose is present in a composition, the mannoheptulose is less than about 5%, or less than about 2%, or from about 0.0001% to about 0.5% of the mannoheptulose, all by weight of the composition.

The level of oxidized glutathione in the blood subsequent to administration of a composition comprising a mannoheptulose is from about 0 μM to about 500 μM, from about 5 μM to about 300 μM, from about5 μM to about 150 μM, from about 10 μM to about 100 μM, as measured by the method described herein.

The level of reduced glutathione in the blood subsequent to administration of a composition comprising mannoheptulose is from about 0 μM to about 4000 μM, from about 10 μM to about 3000 μM, from about 20 μM to about 2500 μM, from about 40 μM to about 2000 μM, as measured by the method described herein.

The level of total glutathione (total GSHₜ) in the blood subsequent to administration of a composition comprising mannoheptulose is from about 0 μM to about 4000 μM, from about 10 μM to about 3000 μM, from about 20 μM to about 2500 μM, from about 40 μM to about 2000 μM, as measured by the method described herein.

A ratio of reduced glutathione to oxidized glutathione in the blood subsequent to administration of the mannoheptulose is from about 0.1:1 to about 500:1, from about 0.1:1 to about 250:1, from about 1:1 to about 100:1, from about 1:1 to about 80:1.

### Avocado

The method of the present invention can comprise administering to a mammal a composition that can comprise avocado. The avocado affects the level and ratio of oxidized glutathione and reduced glutathione present in the blood of a mammal. The decrease of the oxidized glutathione and maintenance of the level reduced glutathione decreases inflammation and oxidative stress subsequent to a mammal ingesting a composition comprising avocado.

The level of an oxidized glutathione in the blood subsequent to administration of a composition comprising avocado is from about 0 μM to about 500 μM, from about 5 μM to about 300 μM, from about 5 μM to about 150 μM, from about 10 μM to about 100 μM, as measured by the method described herein.

The level of reduced glutathione in the blood subsequent to administration of a composition comprising avocado is from about 0 μM to about 4000 μM, from about 10 μM to about 3000 μM, from about 20 μM to about 2500 μM, from about 40 μM to about 2000 μM, as measured by the method described herein.

The level of total glutathione (total GSHT) in the blood subsequent to administration of a composition comprising avocado is from about 0 μM to about 4000 μM, from about 10 μM to about 3000 μM, from about 20 μM to about 2500 μM, from about 40 μM to about 2000 μM, as measured by the method described herein.

A ratio of reduced glutathione to oxidized glutathione in the blood subsequent to administration of avocado is from about is from about 0.1:1 to about 500:1, from about 0.1:1 to about 250:1, from about 1:1 to about 100:1, from about 1:1 to about 80:1.

An avocado (also commonly referred to as alligator pear, aguacate, or palta) contains unusually enriched sources of mannoheptulose, as well as related sugars and other carbohydrates. Avocado is a sub-tropical evergreen tree fruit, growing most successfully in areas of California, Florida, Hawaii, Guatemala, Mexico, the West Indies, South Africa, and Asia.

Nonlimiting examples of species of avocado that can be used in the present invention include, for example, Persea Americana and Persea nubigena, including all cultivars within these illustrative species. Cultivars may include ‘Anaheim,’ ‘Bacon,’ ‘Creamhart,’ ‘Duke,’ ‘Fuerte,’ ‘Ganter,’ ‘Gwen,’ ‘Hass,’ ‘Jim,’ ‘Lula,’ ‘Lyon,’ ‘Mexicola,’ ‘Mexicola Grande,’ ‘Murrieta Green,’ ‘Nabal,’ ‘Pinkerton,’ ‘Queen,’ ‘Puebla,’ ‘Reed,’ ‘Rincon,’ ‘Ryan,’ ‘Spinks,’ ‘Topa Topa,’ ‘Whitsell,’ ‘Wurtz,’ and ‘Zutano.’ The fruit of the avocado is particularly preferred for use herein, which may contain the pit or wherein the pit is removed or at least partially removed. Fruit from Persea Americana is particularly preferred for use herein, as well as fruit from cultivars which produce larger fruits (e.g., about 12 ounces or more when the fruit is mature), such as Anaheim, Creamhart, Fuerte, Hass, Lula, Lyon, Murrieta Green, Nabal, Queen, Puebla, Reed, Ryan, and Spinks.

The dose of avocado given to a mammal, on a daily basis, is from about 100 mg/kg to about 200 g/kg, from about 200 mg/kg to about 20 g/kg, from about 400 mg/kg to about 10 g/kg, wherein (as will be commonly understood in the art) the “mg” refers to level of avocado and the “kg” refers to kilograms of the mammal or from about 0.1 gram to about 200 gram of avocado per kilogram of the mammal. When avocado is present in a composition, avocado is less than about 50%, or less than about 25%, or from about 0.0001% to about 5% of avocado, all by weight of the composition. The level of avocado may be determined by one of ordinary skill in the art based on a variety of factors, for example, the form of the composition (e.g., whether a dry composition, moist composition, wet composition, or supplement, or any other form or mixture thereof). The ordinarily skilled artisan will be able to utilize the preferred optimal doses, and use these to determine the optimal level of component within a given composition.

Advantageously, mannoheptulose or any other component may be present in the recited compositions as a component of plant matter such as avocado, or other enriched source of mannoheptulose including but not limited to alfalfa, fig, or primrose. The plant matter may include the fruit, seed (or pit), branches, leaves, or any other portion of the relevant plant or combination thereof. Additionally, plant matter from alfalfa, fig, or primrose and the like is also reported to provide relatively high levels of mannoheptulose. Alfalfa is also referred to as Medicago sativa. Fig, or Ficus carica (including Cluster fig or Sycamore fig, for example) may also be used, as well as primrose or Primula officinalis.

The mannoheptulose or any other component can be extracted from the plant matter and or avocado to form a plant extract or component extract or avocado extract and then utilized in the composition of the present invention.

When an extract of plant matter is utilized in a composition herein, the component will be present from about 1% to about 99% of the component extract, from about 5% to about 75% of the component extract, from about 10% to about 50% of the component extract, all by weight of the extract.

When an avocado extract is utilized in a composition herein, the component will be present from about 1% to about 99% of the component extract, from about 5% to about 75% of the component extract, from about 10% to about 50% of the component extract, all by weight of the extract.

When an extract of plant matter is mannoheptulose and then utilized in a composition herein, the mannoheptulose will be present from about 1% to about 99% of mannoheptulose, from about 5% to about 75% of the mannoheptulose, from about 10% to about 50% of the mannoheptulose, all by weight of the extract.

When an extract of avocado is mannoheptulose and then utilized in a composition herein, the mannoheptulose will be present from about 1% to about 99% of mannoheptulose, from about 5% to about 75% of the mannoheptulose, from about 10% to about 50% of the mannoheptulose, all by weight of the extract.

The dose of mannoheptulose used when obtained from a plant or avocado extract that is given to a mammal, on a daily basis, is from about 0.1 mg/kg to about 1000 mg/kg, from about 2 mg/kg to about 100 mg/kg, from about 2 mg/kg to about 5 mg/kg, wherein (as will be commonly understood in the art) the “mg” refers to level of the mannoheptulose and the “kg” refers to kilograms of the mammal or from about 0.001 gram to about 1 gram of mannoheptulose per kilogram of the mammal. When mannoheptulose obtained from a plant extract or avocado extract is present in a composition, the mannoheptulose is less than about 5%, or less than about 2%, or from about 0.0001% to about 0.5% of the mannoheptulose, all by weight of the composition. The level of mannoheptulose may be determined by one of ordinary skill in the art based on a variety of factors, for example, the form of the composition (e.g., whether a dry composition, moist composition, wet composition, or supplement, or any other form or mixture thereof). The ordinarily skilled artisan will be able to utilize the preferred optimal doses, and use these to determine the optimal level of component within a given composition.

The level of oxidized glutathione in the blood subsequent to administration of a composition comprising an extract of mannoheptulose obtained from plant matter extract or avocado extract is from about 0 μM to about 500 μM, from about 5 μM to about 300 μM, from about 5 μM to about 150 μM, from about 10 μM to about 100 μM, as measured by the method described herein.

The level of reduced glutathione in the blood subsequent to administration of a composition comprising an extract of mannoheptulose obtained from plant matter extract or avocado extract is from 0 μM to about 4000 μM, from about 10 μM to about 3000 μM, from about 20 μM to about 2500 μM, from about 40 μM to about 2000 μM, as measured by the method described herein.

The level of total glutathione (total GSHₜ) in the blood subsequent to administration of a composition comprising an extract of mannoheptulose obtained from plant matter extract or avocado extract is from 0 μM to about 4000 μM, from about 10 μM to about 3000 μM, from about 20 μM to about 2500 μM, from about 40 μM to about 2000 μM, as measured by the method described herein.

A ratio of reduced glutathione to oxidized glutathione in the blood subsequent to administration of an extract of mannoheptulose from plant matter and/or avocado extract is from about 0.1:1 to about 500:1, from about 0.1:1 to about 250:1, from about 1:1 to about 100:1, from about 1:1 to about 80:1.

### Compositions

It is anticipated that the glucose anti-metabolite or avocado or mannoheptulose or avocado extract or plant matter extract described in the present invention can be added to any composition adapted for administration to a mammal.

Typical formulae for compositions are well known in the art. In addition to proteinaceous and farinaceous materials, the compositions of the invention generally may include vitamins, minerals, and other additives such as flavorings, preservatives, emulsifiers and humectants. The nutritional balance, including the relative proportions of vitamins, minerals, protein, fat and carbohydrate, is determined according to dietary standards known in the veterinary and nutritional art.

Nonlimiting examples of dry compositions may optionally contain on a dry matter basis, from about 1% to about 50% crude protein, from about 0.5% to about 25% crude fat, from about 1% to about 10% supplemental fiber, all by weight of the composition. The dry composition may have a total moisture content from about 1% to about 30% moisture. Alternatively, a dry composition may contain on a dry matter basis, from about 5% to about 35% crude protein, from about 5% to about 25% crude fat, from about 2% to about 8% supplemental fiber, all by weight of the composition. The dry composition may have a total moisture content from about 2% to about 20% moisture. Alternatively, the dry composition contains on a dry matter basis, a minimum protein level of about from about 9.5% to about 35%, a minimum fat level of from about 8% to about 20%, a minimum supplemental fiber level of from about 3% to about 7%, all by weight of the composition. The dry animal composition may also have a minimum metabolizable energy level of about 3.5 Kcal/g. The dry composition may have a total moisture content from about 3% to about 10%, Nonlimiting examples of a semi-moist composition may optionally contain on a dry matter basis, from about 0.5% to about 50% crude protein, from about 0.5% to about 25% crude fat, from about 0.5% to about 15% supplemental fiber, all by weight of the composition. The semi-moist composition may have a total moisture content from about 30% to about 50% moisture. Alternatively, the semi-moist compositions may contain on a dry matter basis, from about 5% to about 35% crude protein, from about 5% to about 25% crude fat, from about 1% to about 5% supplemental fiber, and all by weight of the composition. The semi-moist composition may have a total moisture content from about 35% to about 45% moisture. Alternatively, the semi-moist composition may have on a dry matter basis, a minimum protein level of about from about 9.5% to about 22%, a minimum fat level of from about 8% to about 13%, a minimum supplemental fiber level of from about 2% to about 3%, all by weight of the composition. The semi-moist composition may have a total moisture content from about 38% to about 42%. The semi-moist composition may also have a minimum metabolizable energy level of about 3.5 Kcal/g and from about 0.1% to about 20% ash, and from about 0.001% to about 5.0% taurine.

Nonlimiting examples of a moist composition may optionally contain on a dry matter basis, from about 0.5% to about 50% crude protein, from about 0.5% to about 25% crude fat, from about 0.01% to about 15% supplemental fiber, all by weight of the composition. The moist composition may have a total moisture content from about 50% to about 90% moisture. Alternatively, the moist compositions may contain on a dry matter basis, from about 5% to about 35% crude protein, from about 5% to about 25% crude fat, from about 0.05% to about 5% supplemental fiber, all by weight of the composition. The moist composition may have a total moisture content from about 60% to about 85% moisture. Alternatively, a moist animal composition may contain on a dry matter basis, a minimum protein level of about from about 9.5% to about 22%, a minimum fat level of from about 8% to about 13%, a minimum supplemental fiber level of from about 0.1% to about 3%, all by weight of the composition. The moist composition may have a total moisture content from about 65% to about 80%. The moist composition may also have a minimum metabolizable energy level of about 1.0 Kcal/g and from about 0.1% to about 20% ash, and from about 0.001% to about 5.0% taurine.

In one embodiment of the present invention, the composition is a composition, whether dry, moist, semi-moist or otherwise, that comprises on a dry matter basis, from about 5% to about 50%, alternatively 20% to about 50% of animal-derived ingredients, by weight of the composition. Non-limiting examples of animal-derived ingredients include chicken, beef, pork, lamb, turkey (or other animal) protein or fat, egg, fishmeal, and the like.

Where the composition is in the form of a gravy, the composition may comprise at least 10% of a broth, or stock, non-limiting examples of which include vegetable beef, chicken or ham stock. Typical gravy compositions may comprise on a dry matter basis, from about 0.5% to about 5% crude protein, and from about 2% to about 5% crude fat.

Where the composition is in the form of a supplement composition such as biscuits, chews, and other treats, the supplement may comprise, on a dry matter basis, from about 20% to about 60% protein, from about 22% to about 40% protein, by weight of the supplement composition. As another example, the supplement compositions may comprise, on a dry matter basis, from about 5% to about 35% fat, or from about 10% to about 30% fat, by weight of the supplement composition. Compositions and supplement compositions intended for use by animals such as cats or dogs are commonly known in the art.

### Optional Ingredients

The composition of the present invention can further comprise a wide range of other optional ingredients.

Nonlimiting examples of additional components include animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, textured wheat protein, textured soy protein, textured lupin protein, textured vegetable protein, breading, comminuted meat, flour, comminuted pasta, water, and combinations thereof.

Nonlimiting examples of optional ingredients can include at least one vegetable. Nonlimiting examples of vegetables include carrots, peas, potatoes, cabbage, celery, beans, corn, tomatoes, broccoli, cauliflower, leeks and combinations thereof.

Also useful herein, as an optional ingredient, is a filler. The filler can be a solid, a liquid or packed air. The filler can be reversible (for example thermo-reversible including gelatin) and/or irreversible (for example thermo-irreversible including egg white). Nonlimiting examples of the filler include gravy, gel, jelly, aspic, sauce, water, air (for example including nitrogen, carbon dioxide, and atmospheric air), broth, and combinations thereof. Nonlimiting examples of colorants include, but are not limited to, synthetic or natural colorants, and any combination thereof. When present the colorants are from about 0.0001% to about 5%, from about 0.001% to about 1%, from about 0.005% to about 0.1%, on a dry matter basis, of said colorant.

Additionally, probiotic microorganisms, such as Lactobacillus or Bifidobacterium species, for example, may be added to the composition or the animal food compositions themselves.

Also useful herein, as an optional ingredient, is at least one fruit. Nonlimiting examples include tomatoes, apples, pears, peaches, cherries, apricots, plums, grapes, oranges, grapefruit, lemons, limes, cranberries, raspberries, blueberries, watermelon, cantelope, mushmellon, honeydew melon, strawberries, banana, and combinations thereof.

The composition may contain other active agents such as long chain fatty acids and zinc. Suitable long chain fatty acids include alpha-linoleic acid, gamma linolenic acid, linoleic acid, eicosapentanoic acid, and docosahexanoic acid. Fish oils are a suitable source of eicosapentanoic acids (EPA) and docosahexanoic acid (DHA). The DHA level is at least about 0.05%, alternatively at least about 0.1%, alternatively at least about 0. 15% of the animal food composition, all on a dry matter basis. The EPA level is at least about 0.05%, alternatively at least about 0.1%, alternatively at least about 0.15% of the animal food composition, all on a dry matter basis.

The compositions of the present invention may further comprise a source of carbohydrate. Grains or cereals such as rice, corn, milo, sorghum, barley, wheat, and the like are illustrative sources.

The compositions may also contain other materials such as dried whey and other dairy by products.

### Optional Processes for Preparing the Compositions of the Present Invention

The compositions may be prepared by any of a variety of processes including, but not limited to, optional processes described herein. Disclosed herein are optional processes for preparing the present inventive compositions. The ordinarily skilled artisan will understand, however, that the compositions are not limited by the following described processes. A process for preparing the present compositions may comprise:
(a) providing plant matter;
(b) combining the plant matter with an aqueous solution and optionally with an enzyme, further optionally with heating, to provide a digested plant mixture;
(c) optionally separating any fractions present in the digested plant mixture, if any, to provide a carbohydrate extract;
(d) concentrating the digested plant mixture to enhance the concentration of carbohydrate therein; and
(e) combining the digested plant mixture with one or more composition components.

The plant matter may be any portion or whole of the plant, such as the leaves, fruit, seed or pit. In one optional process herein, avocado is provided, and the process may commence with whole avocado fruit, including the pit or devoid (or partially devoid) of the pit. If the plant matter which is provided contains a pit, or partial pit, the pit or portion thereof may be optionally removed prior to further processing. Alfalfa, fig, or primrose and the like may be similarly processed.

Additionally, in the production of a digested plant mixture can comprise combination of the plant matter with an aqueous solution, such as water, to assist with maceration of the plant into manageable constituents. Optionally but preferably, an enzyme having cellulose or pectin activity, or any combination thereof (such as a cellulase, hemicellulase, or pectinase) is included to assist with such maceration, including to assist with dissolution and release of carbohydrates via cell wall disruption. The utility of such an enzymatic treatment may be enhanced through heating during such maceration, such as from above ambient temperature to about 120° C., or to about 100° C., or from about 60° C. to about 120° C., or from about 60° C. to about 100° C. Agitation is further preferably utilized, typically for up to about 24 hours, but dependent upon the batch under processing. In one embodiment, the pH is controlled such to preserve enzyme activity, often in the range of pH from about 4 to about 6, preferably in the range of pH from about 5 to about 6. As such, depending upon such factors as ripeness of plant matter, quality of process aqueous solution (such as water added for process, for example), and the like, amounts of acid or base may be desirable as will be appreciated by one of ordinary skill in the art. Optionally, to assist with deactivation of the enzymes present, heating may be increased at the time of, or after, initial heating and agitation to form the digested plant mixture. Water is optionally heated to processing temperatures prior to the addition of the plant matter. Heat may be applied by a jacketed tank where low pressure steam is utilized. The digested plant mixture may result in fractions which may be separated in accordance with common techniques. For example, fractions present in the digested plant mixture may be separated by filtration to provide the carbohydrate extract as the resulting filtrate, with the filter cake being discarded. Other methods may include, but not be limited to, gravimetric, centrifugal, other filtrations, or combinations thereof.

The carbohydrate extract may then be concentrated, optionally utilizing at least one concentration method selected from the group consisting of heating, vacuum drying, evaporation, refractance window drying, freeze drying, spray drying, any other useful technique, or any combination of the foregoing. In one embodiment, at least one technique such as refractance window drying is used.

Once concentrated, the carbohydrate extract may be utilized in a composition of the present invention. In one embodiment herein, the present processes result in preferred yields of mannoheptulose or other components, based on the starting mass of the plant matter (e.g., avocado). In one embodiment, the yield of mannoheptulose present in the carbohydrate extract subsequent to concentration is less than about 20%, or from about 0.1% to about 10%, or from about 1% to about 7%, based on the starting mass of the plant matter. In another embodiment, the yield of the carbohydrate extract subsequent to concentration is less than about 30%, or from about 5% to about 25%, or from about 8% to about 20%, based on the starting mass of the plant matter. Of course, even higher yields may be desirable, and lower yields may also be acceptable.

### Reduced Glutathione (GSH) and Oxidized Glutathione (GSSG) Method

The method measures the endogenous level of reduced glutathione (GSH) and oxidized glutathione (GSSG) in a sample of whole blood. The method can be used to determine the ratio of GSH to GSSG in a whole blood sample. Additionally, the method can be used to determine the total (GSHT) which includes reduced GSH in combination with reduced GSH derived from the conversion of GSSG to two molecules of reduced GSH. For Example, kits obtained from OXIS Health Product Inc. can be used to perform the method disclosed herein.

### Materials

Assay Buffer Na·PO4 with EDTA, dry powder.
GSSG Buffer Na·PO4 with EDTA, 150 mL.
Enzyme Glutathione reductase (GR) in Na·PO4 with EDTA, 40 mL.
NADPH â-Nicotinamide adenine dinucleotide phosphate with Tris base and mannitol, 6 vials lyophilized powder.
Scavenger 1-Methyl-2-vinyl-pyridium trifluoromethane sulfonate (M2VP) in HCl, 2 mL which functions to bind endogenous GSH in the sample.
Chromogen 5,5′-Dithiobis-(2-nitrobenzoic acid) (DTNB) in Na·PO4 with EDTA, with ethanol, 40 mL.
Standards GSSG in KPO4 buffer with EDTA, 2.0 mL each. Each GSSG molecule is equivalent to two GSH molecules; therefore, the values are expressed as μM GSH:

| | GSSG, μM | 2 GSH, μM |
|---|---|---|
| 1 | 0.000 | 0.00 |
| 2 | 0.050 | 0.10 |
| 3 | 0.125 | 0.25 |
| 4 | 0.250 | 0.50 |
| 5 | 0.750 | 1.50 |
| 6 | 1.500 | 3.00 |

Unicam UV1 Spectrophotometer
Centrifuge capable of 2000×g for 10 minutes at 8° C.
Methacrylate cuvettes with a 10 mm optical path length and 1.5 ml volume
Disposable tubes and stoppers (glass or polypropylene).
Pipettes, preferably adjustable capable of accurately pipetting 10, 50, 100, 200, 700 and 3000 μL.
Balance
Metaphosphoric acid (Sigma M-5043)

### Reagent Storage and Handling

When not in use, place the bottles at 4° C.

### Procedure

### Reagent Preparation

NADPH: Just prior to use, reconstitute the lyophilized NADPH Reagent with 7.5 mL of Assay Buffer. The reconstituted NADPH Reagent is stable for 6 hours at room temperature.
Assay Buffer: Reconstitute the dry powder with 650 mL of deionized water. The reconstituted reagent is stable at 4° C. for the life of the kit.
5% Metaphosphoric Acid: Prepare fresh. Weigh 1 gram MPA and dissolve in 20 mL deionized water.
MPA and NADPH reagents are intended for same day use following reconstitution.

### Preparing GSSG Standard

Standards are ready to use.

### Sample Preparation

The sample preparation for whole blood is described below.

### Whole Blood

Pipetting. Use positive displacement pipetting techniques for total GSHₜ and GSSG method with whole blood.

Freezing Step. The freezing step serves to lyse the red blood cell and maximize the concentration of GSSG in the sample.

Frozen Samples. Blood samples that have been frozen without prior treatment with the Scavenging Reagent are not suitable for the GSSG method.

Total GSHₜ Linearity. Because GSH is at high concentrations in whole blood, approximately 1 mM of the whole blood sample will be diluted 488 times in order to maintain linearity of the reaction rate.
Sample Stability. Glutathione (GSH) and oxidized glutathione (GSSG) are relatively stable in intact “resting” cells for up to 24 hr at 4° C., Blood samples should be treated with M2VP as soon as possible and frozen immediately.

### GSSG Sample Determination

1. Add 10 μL M2VP to a microcentrifuge tube to bind endogenous GSH.
2. Carefully add 100 μL whole blood that has been properly prepared to the bottom of the centrifuge tube.
3. Mix gently with Vortex Genie setting 8.
4. Freeze the sample at −70° C.
5. Thaw the sample and immediately mix ( Vortex Genie at setting 8), incubate at room temperature for 2-10 minutes.
6. Add 290 μL cold 5% MPA to the tube (¼ dilution of GSSG sample).
7. Vortex the GSSG sample for 15-20 seconds.
8. Centrifuge at 2000×g for 10 minutes at 8° C.
9. Add 50 μL MPA extract to 700 μL GSSG buffer ( 1/15 dilution of the acid extract).
10. Place the diluted extract on ice. (Final sample dilution is 1/60).
   GSSG Blank determination
1. Add 50 μL MPA to 700 μL GSSG buffer ( 1/15 dilution of the acid extract).
2. Place the diluted MPA on ice until use (Final sample dilution is 1/60).

### Total GSHₜ Sample

1. Carefully add 50 μL of whole blood to the bottom of a microcentrifuge tube.
2. Freeze the sample at −70° C.
3. Thaw the sample and immediately mix with Vortex Genie setting 8.
4. Add 350 μL cold 5% MPA to the tube (⅛ dilution of total GSHₜ sample).
5. Vortex the total GSHₜ sample for 15-20 seconds.
6. Centrifuge at 2000×g for 10 minutes at 8° C.
7. Add 50 μL MPA extract to 3 mL Assay Buffer ( 1/61 dilution of the acid extract).
8. Place diluted extract on ice. (Final sample dilution is 1/488).
   Method for GSSG and total GSHₜ Determinations
1. Add 200 μL of GSSG standards, GSSG blank and either GSSG or Total GSHₜ blood samples to the cuvettes depending on which one you are measuring first.
2. Add 200 μL of Chromogen to each cuvette.
3. Add 200 μL of Enzyme to each cuvette.
4. Mix and incubate at room temperature for 5 minutes.
5. Add 200 μL of NADPH to each cuvette.
6. Zero the Spectrophotometer on water.
7.Record the change of absorbance for each standard and sample on the Spectrophotometer at 412 nm 0, 30, 60, 90, 120 and 150 seconds.

### Calculations

The calculation of the GSH and GSSG concentrations and the GSH/GSSG ratio requires five steps:
1) Determination of the reaction rate,
2) Construction of calibration curves,
3) Calculation of the analyte concentrations (GSSG and total GSHₜ),
4) Calculation of the GSH concentration; and
5) Calculation of the GSH/GSSG ratio.

### Reaction Rate Determination

The change in absorbance at 412 nm is a linear function of the total GSHₜ concentration in the reaction mixture, is described by the following equation of a line:

A₄₁₂=slope×Minutes+intercept

where the slope of the regression equation is equal to the reaction rate. The intercepts for these rate curves are ignored because they are dependent on the DTNB background and the time interval between the addition of the NADPH (reaction start) and the first recorded A₄₁₂.

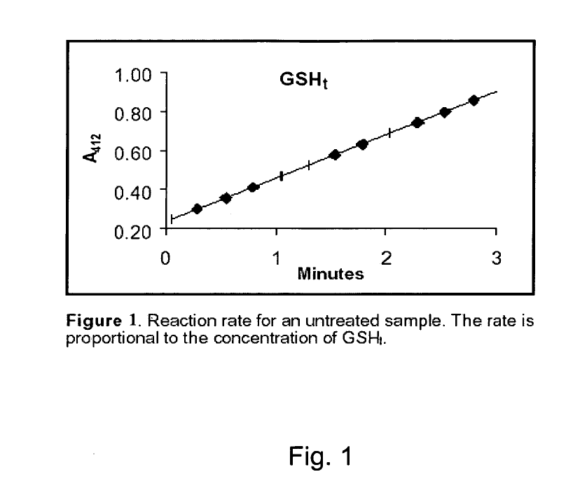

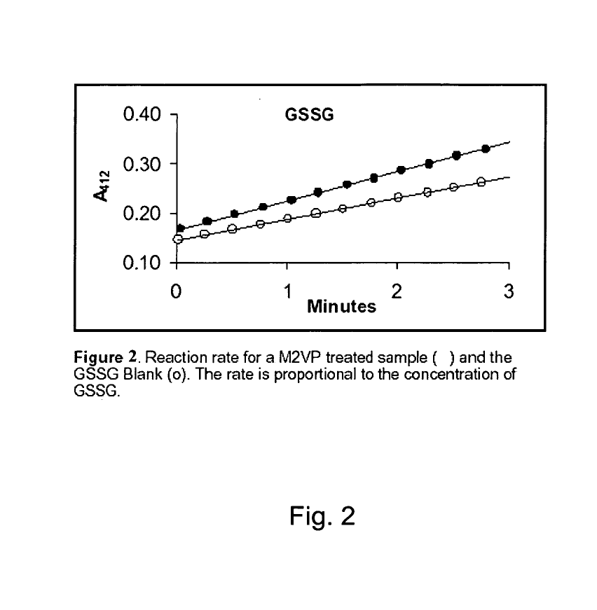

In the examples below using the GSSG and Total GSHₜ samples, linear regression gave the following equation of the line for GSH derived from the total GSHt (FIG. 1) and GSH derived only from GSSG (FIG. 2):

Total GSHt: A412=0.2209×Minutes+0.2363 with an r2 value of 1.0000. Therefore, the rate for the total GSHt sample is 0.2209 A412/min.
GSSG: A412=0.05938×Minutes+0.1651 with an r2 value of 0.9999. Therefore, the rate for the GSSG sample is 0.05938 A412/min.
GSSG BLANK: A412=0.04238×Minutes+0.1454 with an r2 value of 0.9999. Therefore, the rate for the GSSG Blank is 0.04238 A412/min.

### Calibration Curves

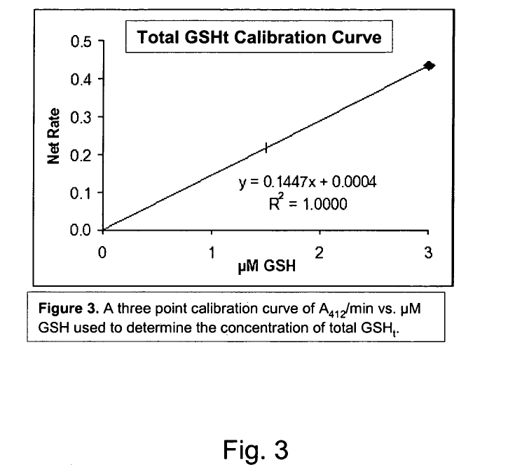

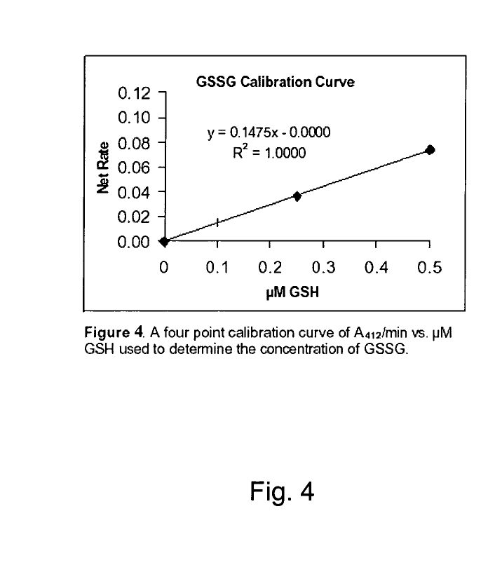

The GSH/GSSG-412 assay uses a six-point standard curve for both total GSHt and GSSG determinations. The Net Rate is the difference between the rate at each concentration of total GSHt and the Blank rate. Because the concentration of GSSG is much lower in the reaction mixture compared to total GSHt, it is recommended that selected data ranges from the calibration curve be plotted separately. For total GSHt, perform linear regression on a three-point curve using the 0, 1.50 and 3.00 μM GSH data points, see FIG. 3. In the case of GSSG, use the 0, 0.10, 0.25, and 0.50 μM GSH data points, see FIG. 4.

### Total GSHt, GSSG and GSH Concentrations

The general form of the regression equation describing the calibration curve is:

Net Rate=slope×total GSHt+intercept

Therefore, the total GSHt calibration curve is used to calculate both the total GSHt and GSSG concentrations in the sample:

Total GSHt=(Net Rate−Intercept)/Slope×Dilution Factor

GSSG sample:

GSSG=(Net Rate−Intercept)/Slope×Dilution Factor

For example, from FIG. 1, the net rate of change for the total GSHt sample is 0.2209-0.0423 or 0.1786 A412/min. Using the calibration curve parameters from FIG. 3, the total GSHt can be calculated as follows:

Total GSHt=(0.1786−0.0004)/0.1447×488=601.0 μM

From FIG. 2, similarly, the rate of change for the oxidized GSSG sample is 0.05938-0.04238 or 0.0170 A412/min. Using the calibration curve parameters from FIG. 4, the oxidized GSSG concentration can be calculated as shown below.

Oxidized GSSG=(0.01700−0.0000)/0.1475×30=3.448 uM

Note that the dilution factor correction is 30, which accounts for the original 60-fold dilution divided by the generation of 2 molecules of GSH per 1 molecule of oxidized GSSG (60/2=30).

### GSH Concentration

The concentration of reduced GSH in the sample is calculated by determining the difference between GSH derived from total GSHt and oxidized GSSG concentrations:

Reduced GSH=Total GSHt−(2×Oxidized GSSG)

Continuing the above Example, the concentration of GSH is:

Reduced GSH=601.0−6.8960=594.104 uM

### GSH/GSSG Ratio

The ratio of GSH/GSSH Ratio is then calculated by dividing the difference between the concentration of GSH by the concentration of GSSG.

GSH:GSSG ratio=GSH concentration /GSSG concentration

Continuing the above example, the ratio of GSH:GSSG ratio is:

GSH:GSSG ratio=594.104/3.448=172.3:1 ratio

### Total Moisture Content Method

The method involves the analysis of the total moisture content in the composition. The analysis is based on the procedure outlined in AOAC method 930.15 and AACC method 44-19.

A composition sample is prepared by taking one unit volume, for example, 375 gram of the composition, and homogenizing in a food processor to a uniform consistency like a paste. A composition larger than 375 gram would be subdivided to create equal and representative fractions of the whole such that a 375 gram sample is obtained.

The paste of the composition is individually sampled in triplicate at a volume less than or equal to 100 ml and placed individually sealed in a 1 00ml Nasco Whirl-Pak® (Fort Atkinson, WI 53538-0901). During the process of sealing the Whirl-Pak®, excess air is evacuated manually from the container just prior to final closure thereby minimizing the container headspace. The Whirl-Pak® is closed per manufacturer's instructions—tightly folding the bag over three (3) times and bending the tabs over 180 degrees.

All samples are refrigerated at 6° C. for less than 48 h prior to moisture analysis.

For total moisture analysis, the tare weight of each moisture tin and lid are recorded to 0.0001 g. Moisture tins and lids are handled using dry and clean forceps. Moisture tins and lids are held dry over desiccant in a sealed desiccator. A Whirl-Pak® containing a sample is unfolded and a 2.0000+/−0.2000 gram sample is weighed into the uncovered moisture tin. The weight of the sample in the moisture tin is recorded. The lid is placed atop the moisture tin in an open position to allow moisture loss but contain all other material during air oven drying. The lid and moisture tin loaded with sample are placed in an air oven operating at 135° C. for 6 h. Time is tracked using a count-down timer.

After drying, the tin is removed from the oven and the dried lid is placed atop the tin using forceps. The covered moisture tin with dried sample is placed immediately in a desiccator to cool. The sealed desiccator is filled below the stage with active desiccant. Once cool to room temperature, the covered moisture tin with dried sample is weighed to 0.0001 g and weight recorded. The total moisture content of each sample is calculated using the following formula:

Total Moisture Content (%)=100−(weight of tin, lid and sample after drying−empty tin and lid weight)×100/initial sample weight.

### EXAMPLES

The following examples further describe and demonstrate embodiments within the scope of the invention. The examples are given solely for the purpose of illustration and are not to be construed as limitations of the present invention, as many variations thereof are possible without departing from the spirit and scope of the invention. All of the following examples are compositions that are utilized by a mammal.

### Examples 1-72:

Dry compositions

| | Percentage % on dry matter basis (w/w) | | | | | |
|---|---|---|---|---|---|---|
| Ingredient | Example 1 | Example 2 | Example 3 | Example 4 | Example 5 | Example 6 |
| Protein products and | 26.4000 | 42.0000 | 45.3000 | 55.8000 | 56.0000 | 37.0000 |
| meals | | | | | | |
| Cereal grains | 64.0500 | 43.0500 | 37.6500 | 27.4500 | 26.7500 | 45.9500 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 8.0000 | 7.0000 |
| Avocado Extract | 0.0500 | 0.0500 | 0.0500 | 0.0500 | 0.0500 | 0.0500 |
| | Percentage % on dry matter basis (w/w) | | | | | |
| | | | | Example | Example | Example |
| Ingredient | Example 7 | Example 8 | Example 9 | 10 | 11 | 12 |
| Protein products and | 26.4000 | 42.0000 | 45.3000 | 55.8000 | 56.0000 | 37.0000 |
| meals | | | | | | |
| Cereal grains | 64.0975 | 43.0975 | 37.6975 | 27.4975 | 26.7975 | 45.9975 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 8.0000 | 7.0000 |
| Avocado Extract | 0.0025 | 0.0025 | 0.0025 | 0.0025 | 0.0025 | 0.0025 |
| | Percentage % on dry matter basis (w/w) | | | | | |
| | Example | Example | Example | Example | Example | Example |
| Ingredient | 13 | 14 | 15 | 16 | 17 | 18 |
| Protein products and | 21.4000 | 37.0000 | 40.2000 | 50.7000 | 51.0000 | 32.0000 |
| meals | | | | | | |
| Cereal grains | 59.1000 | 38.1000 | 32.8000 | 22.6000 | 21.8000 | 41.0000 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 8.0000 | 7.0000 |
| Avocado Extract | 10.0000 | 10.0000 | 10.0000 | 10.0000 | 10.0000 | 10.0000 |
| | Percentage % on dry matter basis (w/w) | | | | | |
| | Example | Example | Example | Example | Example | Example |
| Ingredient | 19 | 20 | 21 | 22 | 23 | 24 |
| Protein products and | 25.9000 | 48.0000 | 45.0000 | 54.9000 | 56.0000 | 50.0000 |
| meals | | | | | | |
| Cereal grains | 63.6000 | 36.1000 | 37.0000 | 27.4000 | 26.8000 | 32.0000 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Avocado | 1.0000 | 1.0000 | 1.0000 | 1.0000 | 1.0000 | 1.0000 |
| | Percentage % on dry matter basis (w/w) | | | | | |
| | Example | Example | Example | Example | Example | Example |
| Ingredient | 25 | 26 | 27 | 28 | 29 | 30 |
| Protein products and | 25.9000 | 48.0000 | 45.0000 | 54.9000 | 56.0000 | 50.0000 |
| meals | | | | | | |
| Cereal grains | 39.6000 | 12.1000 | 13.0000 | 3.4000 | 2.8000 | 8.0000 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Avocado | 25.0000 | 25.0000 | 25.0000 | 25.0000 | 25.0000 | 25.0000 |
| | Percentage % on dry matter basis (w/w) | | | | | |
| | Example | Example | Example | Example | Example | Example |
| Ingredient | 31 | 32 | 33 | 34 | 35 | 36 |
| Protein products and | 26.4000 | 48.5000 | 45.5000 | 55.4000 | 56.5000 | 50.5000 |
| meals | | | | | | |
| Cereal grains | 64.0500 | 36.5500 | 37.4500 | 27.8500 | 27.2500 | 32.4500 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Avocado | 0.0500 | 0.0500 | 0.0500 | 0.0500 | 0.0500 | 0.0500 |
| | Percentage % on dry matter basis (w/w) | | | | | |
| | Example | Example | Example | Example | Example | Example |
| Ingredient | 37 | 38 | 39 | 40 | 41 | 42 |
| Protein products and | 26.4000 | 48.5000 | 45.0000 | 55.4000 | 56.5000 | 50.0000 |
| meals | | | | | | |
| Cereal grains | 64.0800 | 36.5800 | 37.9800 | 27.8800 | 27.2800 | 32.9800 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Mannoheptulose | 0.0200 | 0.0200 | 0.0200 | 0.0200 | 0.0200 | 0.0200 |
| | Percentage % on dry matter basis (w/w) | | | | | |
| | Example | Example | Example | Example | Example | Example |
| Ingredient | 43 | 44 | 45 | 46 | 47 | 48 |
| Protein products and | 26.4000 | 48.4000 | 45.0000 | 55.4000 | 56.5000 | 50.0000 |
| meals | | | | | | |
| Cereal grains | 64.0990 | 36.6990 | 37.9990 | 27.8990 | 27.2990 | 32.9990 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Mannoheptulose | 0.0010 | 0.0010 | 0.0010 | 0.0010 | 0.0010 | 0.0010 |
| | Percentage % on dry matter basis (w/w) | | | | | |
| | Example | Example | Example | Example | Example | Example |
| Ingredient | 49 | 50 | 51 | 52 | 53 | 54 |
| Protein products and | 21.4000 | 43.4000 | 40.0000 | 50.4000 | 51.5000 | 45.0000 |
| meals | | | | | | |
| Cereal grains | 59.1000 | 31.7000 | 33.0000 | 22.9000 | 22.3000 | 28.0000 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Mannoheptulose | 10.0000 | 10.0000 | 10.0000 | 10.0000 | 10.0000 | 10.0000 |
| | Percentage % on dry matter basis (w/w) | | | | | |
| | Example | Example | Example | Example | Example | Example |
| Ingredient | 55 | 56 | 57 | 58 | 59 | 60 |
| Protein products and | 26.4000 | 48.4000 | 45.0000 | 55.4000 | 56.5000 | 50.0000 |
| meals | | | | | | |
| Cereal grains | 64.0800 | 36.6800 | 37.9800 | 27.8800 | 27.2800 | 32.9800 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Glucose | 0.0200 | 0.0200 | 0.0200 | 0.0200 | 0.0200 | 0.0200 |
| Anti-Metabolite | | | | | | |
| | Percentage % on dry matter basis (w/w) | | | | | |
| | Example | Example | Example | Example | Example | Example |
| Ingredient | 61 | 62 | 63 | 64 | 65 | 66 |
| Protein products and | 26.4000 | 48.4000 | 45.0000 | 55.4000 | 56.5000 | 50.0000 |
| meals | | | | | | |
| Cereal grains | 64.0990 | 36.6990 | 37.9990 | 27.8990 | 27.2990 | 32.9990 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Glucose | 0.0010 | 0.0010 | 0.0010 | 0.0010 | 0.0010 | 0.0010 |
| Anti-Metabolite | | | | | | |
| | Percentage % on dry matter basis (w/w) | | | | | |
| | Example | Example | Example | Example | Example | Example |
| Ingredient | 67 | 68 | 69 | 70 | 71 | 72 |
| Protein products and | 21.4000 | 43.5000 | 40.0000 | 50.4000 | 51.5000 | 45.0000 |
| meals | | | | | | |
| Cereal grains | 59.1000 | 31.6000 | 33.0000 | 22.9000 | 22.3000 | 28.0000 |
| Fat | 2.6000 | 5.8000 | 7.0000 | 6.0000 | 6.0000 | 7.0000 |
| Egg product | 3.5000 | 2.0000 | 3.0000 | 2.0000 | 2.0000 | 2.0000 |
| Vitamins | 0.2000 | 0.4000 | 0.6000 | 0.8000 | 0.4000 | 0.4000 |
| Minerals | 0.2000 | 0.8000 | 0.4000 | 0.8000 | 0.8000 | 0.6000 |
| Fiber | 3.0000 | 5.9000 | 6.0000 | 7.1000 | 7.0000 | 7.0000 |
| Glucose | 10.0000 | 10.0000 | 10.0000 | 10.0000 | 10.0000 | 10.0000 |
| Anti-Metabolite | | | | | | |

The dry compositions of Examples 1-72 can be made by first, milling and mixing the cereal grains with protein meal, egg products, vitamins and minerals and fiber sources and avocado or avocado 5 extract or mannoheptulose or glucose anti-metabolite. Then, add the mixed, dried ingredients to the meat products and fat sources. Extrude the ingredients into kibbles. Dry the kibbles. Package the finished product.

### Examples 73-144:

Wet compositions

| | Percentage % on dry matter basis (w/w) | | | | | |
|---|---|---|---|---|---|---|
| | Example | Example | Example | Example | Example | Example |
| Ingredient | 73 | 74 | 75 | 76 | 77 | 78 |
| Protein products and | 82.7000 | 44.9000 | 54.0000 | 65.3000 | 63.4000 | 48.4000 |
| meals | | | | | | |
| Cereal grains | 11.4000 | 43.7000 | 35.9000 | 29.9000 | 30.1000 | 45.7000 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado Extract | 0.2000 | 0.2000 | 0.2000 | 0.2000 | 0.2000 | 0.2000 |
| | Percentage % on dry matter basis (w/w) | | | | | |
| | Example | Example | Example | Example | Example | Example |
| Ingredient | 79 | 80 | 81 | 82 | 83 | 84 |
| Protein products and | 82.7000 | 44.9000 | 54.0000 | 65.4000 | 63.5000 | 48.4000 |
| meals | | | | | | |
| Cereal grains | 11.5900 | 43.8900 | 36.0900 | 29.9900 | 30.1900 | 45.8900 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado Extract | 0.0100 | 0.0100 | 0.0100 | 0.0100 | 0.0100 | 0.0100 |
| | Percentage % on dry matter basis (w/w) | | | | | |
| | Example | Example | Example | Example | Example | Example |
| Ingredient | 85 | 86 | 87 | 88 | 89 | 90 |
| Protein products and | 72.7000 | 35.0000 | 44.1000 | 55.4000 | 53.5000 | 38.5000 |
| meals | | | | | | |
| Cereal grains | 1.6000 | 33.8000 | 25.0000 | 20.0000 | 20.2000 | 35.8000 |
| Fat | 0.0000 | 2.0000 | 2.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado Extract | 20.0000 | 20.0000 | 20.0000 | 20.0000 | 20.0000 | 20.0000 |
| | Percentage % on dry matter basis (w/w) | | | | | |
| | Example | Example | Example | Example | Example | Example |
| Ingredient | 91 | 92 | 93 | 94 | 95 | 96 |
| Protein products and | 80.7000 | 43.0000 | 52.1000 | 63.4000 | 61.5000 | 46.5000 |
| meals | | | | | | |
| Cereal grains | 9.6000 | 41.8000 | 34.0000 | 28.0000 | 28.2000 | 43.8000 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado | 4.0000 | 4.0000 | 4.0000 | 4.0000 | 4.0000 | 4.0000 |
| | Percentage % on dry matter basis (w/w) | | | | | |
| | Example | Example | Example | Example | Example | Example |
| Ingredient | 97 | 98 | 99 | 100 | 101 | 102 |
| Protein products and | 82.6000 | 44.9000 | 54.0000 | 65.3000 | 63.4000 | 48.4000 |
| meals | | | | | | |
| Cereal grains | 11.5000 | 43.7000 | 35.9000 | 29.9000 | 30.1000 | 45.7000 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado | 0.2000 | 0.2000 | 0.2000 | 0.2000 | 0.2000 | 0.2000 |
| | Percentage % on dry matter basis (w/w) | | | | | |
| | Example | Example | Example | Example | Example | Example |
| Ingredient | 103 | 104 | 105 | 106 | 107 | 108 |
| Protein products and | 72.7000 | 35.0000 | 44.1000 | 55.4000 | 53.5000 | 38.5000 |
| meals | | | | | | |
| Cereal grains | 1.6000 | 33.8000 | 26.0000 | 20.0000 | 20.2000 | 35.8000 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Avocado | 20.0000 | 20.0000 | 20.0000 | 20.0000 | 20.0000 | 20.0000 |
| | Percentage % on dry matter basis (w/w) | | | | | |
| | Example | Example | Example | Example | Example | Example |
| Ingredient | 109 | 110 | 111 | 112 | 113 | 114 |
| Protein products and | 82.7000 | 44.9000 | 54.0000 | 65.3000 | 63.4000 | 48.3000 |
| meals | | | | | | |
| Cereal grains | 11.5200 | 43.8200 | 35.0200 | 30.0200 | 30.2200 | 45.9200 |
| Fat | 0.0000 | 2.0000 | 2.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Mannoheptulose | 0.0800 | 0.0800 | 0.0800 | 0.0800 | 0.0800 | 0.0800 |
| | Percentage % on dry matter basis (w/w) | | | | | |
| | Example | Example | Example | Example | Example | Example |
| Ingredient | 115 | 116 | 117 | 118 | 119 | 120 |
| Protein products and | 82.7000 | 44.9000 | 54.0000 | 65.4000 | 63.5000 | 48.4000 |
| meals | | | | | | |
| Cereal grains | 11.5960 | 43.8960 | 36.0960 | 29.9960 | 30.1960 | 45.8960 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Mannoheptulose | 0.0040 | 0.0040 | 0.0040 | 0.0040 | 0.0040 | 0.0040 |
| | Percentage % on dry matter basis (w/w) | | | | | |
| | Example | Example | Example | Example | Example | Example |
| Ingredient | 121 | 122 | 123 | 124 | 125 | 126 |
| Protein products and | 77.7000 | 40.0000 | 49.1000 | 60.4000 | 58.5000 | 43.5000 |
| meals | | | | | | |
| Cereal grains | 6.6000 | 38.8000 | 31.0000 | 25.0000 | 25.2000 | 40.8000 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Mannoheptulose | 10.0000 | 10.0000 | 10.0000 | 10.0000 | 10.0000 | 10.0000 |
| | Percentage % on dry matter basis (w/w) | | | | | |
| | Example | Example | Example | Example | Example | Example |
| Ingredient | 127 | 128 | 129 | 130 | 131 | 132 |
| Protein products and | 82.7000 | 44.9000 | 54.0000 | 65.3000 | 63.4000 | 48.3000 |
| meals | | | | | | |
| Cereal grains | 11.5200 | 43.8200 | 35.0200 | 30.0200 | 30.2200 | 45.9200 |
| Fat | 0.0000 | 2.0000 | 2.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Glucose | 0.0800 | 0.0800 | 0.0800 | 0.0800 | 0.0800 | 0.0800 |
| Anti-Metabolite | | | | | | |
| | Percentage % on dry matter basis (w/w) | | | | | |
| | Example | Example | Example | Example | Example | Example |
| Ingredient | 133 | 134 | 135 | 136 | 137 | 138 |
| Protein products and | 82.7000 | 44.9000 | 54.0000 | 65.4000 | 63.5000 | 48.4000 |
| meals | | | | | | |
| Cereal grains | 11.5960 | 43.8960 | 36.0960 | 29.9960 | 30.1960 | 45.8960 |
| Fat | 0.0000 | 2.0000 | 1.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Glucose | 0.0040 | 0.0040 | 0.0040 | 0.0040 | 0.0040 | 0.0040 |
| Anti-Metabolite | | | | | | |
| | Percentage % on dry matter basis (w/w) | | | | | |
| | Example | Example | Example | Example | Example | Example |
| Ingredient | 139 | 140 | 141 | 142 | 143 | 144 |
| Protein products and | 77.7000 | 40.0000 | 49.1000 | 60.4000 | 58.5000 | 43.5000 |
| meals | | | | | | |
| Cereal grains | 6.6000 | 38.8000 | 30.0000 | 25.0000 | 25.2000 | 40.8000 |
| Fat | 0.0000 | 2.0000 | 2.0000 | 1.0000 | 1.0000 | 2.0000 |
| Egg product | 2.5000 | 2.0000 | 3.0000 | 2.0000 | 2.0000 | 3.4000 |
| Vitamins | 0.1000 | 0.4000 | 0.6000 | 0.8000 | 0.4000 | 0.1000 |
| Minerals | 0.1000 | 0.8000 | 0.4000 | 0.8000 | 0.4000 | 0.2000 |
| Fiber | 3.0000 | 6.0000 | 4.9000 | 0.0000 | 2.5000 | 0.0000 |
| Glucose | 10.0000 | 10.0000 | 10.0000 | 10.0000 | 10.0000 | 10.0000 |
| Anti-Metabolite | | | | | | |

The wet compositions of Examples 73-144 can be made by first drying and milling cereal grains. Mix dried cereal grains, Protein meals, egg product, vitamins, minerals and fiber sources and avocado or avocado extract or mannoheptulose or glucose anti-metabolite. Blend dry ingredients with meat products and fat sources. The mixture is packaged into cans and cooked via retort process to provided finished product. For preformed pieces (chunks in gravy) mixture is extruded, passed through a steam tunnel for preconditioning, cut to desired shape, packaged with added water and retorted to provide safe finished product.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for decreasing inflammation and oxidative stress in a mammal comprising;
administration to a mammal a composition comprising mannoheptulose; and wherein said composition comprises amounts of the mannoheptulose sufficient to increase a ratio of reduced glutathione to oxidized glutathione in the blood of the mammal subsequent to administration of the mannoheptulose.

2. The method of claim 1, wherein a ratio of said reduced glutathione to a oxidized glutathione in the blood subsequent to administration of the mannoheptulose is from about 0.1:1 to about 500:1.

3. The method of claim 1, wherein a ratio of said reduced glutathione to a oxidized glutathione in the blood subsequent to administration of the mannoheptulose is from about from about 0.1:1 to about 250:1.

4. The method of claim 1, wherein a ratio of said reduced glutathione to a oxidized glutathione in the blood subsequent to administration of the mannoheptulose is from about from about 1:1 to about 100:1.

5. The method of claim 1, wherein the level of said oxidized glutathione in the blood subsequent to administration of the mannoheptulose is from about 0 μM to about 500 μM.

6. The method of claim 1, wherein said mannoheptulose is selected from the group consisting of glucose anti-metabolite, plant matter extract, avocado extract, avocado, and combinations thereof.

7. The method of claim 1, wherein the administration is oral.

8. The method of claim 1, wherein the composition comprises less than about 5% of said mannoheptulose, by weight of the composition.

9. The method of claim 1, wherein said composition is selected from the group consisting of pet food, dog food, cat food, treats, chew, biscuits, gravy, sauce, beverage, supplemental water, and combinations thereof.

10. The method of claim 9, wherein the composition is a nutritionally balanced pet food composition.

11. The method of claim 1, further comprising animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, lipids, and combinations thereof.

12. A method for decreasing inflammation and oxidative stress in a mammal comprising;
administration to a mammal a composition comprising a glucose anti-metabolite; and
wherein said composition comprises amounts of the glucose anti-metabolite sufficient to increase the ratio of reduced glutathione to oxidized glutathione in the blood of the mammal subsequent to administration of the glucose anti-metabolite.

13. The method of claim 12, wherein a ratio of said reduced glutathione to a oxidized glutathione in the blood subsequent to administration of the glucose anti-metabolite is from about 0.1:1 to about 500:1.

14. The method of claim 12, wherein a ratio of said reduced glutathione to a oxidized glutathione in the blood subsequent to administration of the glucose anti-metabolite is from about from about 0.1:1 to about 250:1.

15. The method of claim 12, wherein the glucose anti-metabolite is selected from the group consisting of 2-deoxy-D-glucose, 5-thio-D-glucose, 3-O-methylglucose, anhydrosugar, 1,5-anhydro-D-glucitol, 2,5-anhydro-D-mannitol, mannoheptulose, and combinations thereof.

16. The method of claim 12, wherein the composition comprises less than about 5% of said glucose anti-metabolite, by weight of the composition.

17. The method of claim 12, wherein the administration is oral.

18. The method of claim 12, wherein said composition is selected from the group consisting of pet food, dog food, cat food, treats, chew, biscuits, gravy, sauce, beverage, supplemental water, and combinations thereof.

19. The method of claim 12, further comprising animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, lipids, and combinations thereof.

20. A method for decreasing inflammation and oxidative stress in a mammal comprising;
administration to a mammal a composition comprising avocado; and wherein said composition comprises amounts of avocado sufficient to increase the ratio of reduced glutathione to oxidized glutathione in the blood of the mammal subsequent to administration of avocado.

21. The method of claim 20, wherein a ratio of said reduced glutathione to a oxidized glutathione in the blood subsequent to administration of the avocado is from about 0.1:1 to about 500:1.

22. The method of claim 20, wherein the administration is oral.

23. The method of claim 20, wherein the composition comprises less than about 5% of said avocado, by weight of the composition.

24. The method of claim 20, wherein said composition is selected from the group consisting of pet food, dog food, cat food, treats, chew, biscuits, gravy, sauce, beverage, supplemental water, and combinations thereof.

25. The method of claim 20, further comprising animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, lipids, and combinations thereof.

26. A method for decreasing inflammation and oxidative stress in a mammal comprising;
administration to a mammal a composition comprising mannoheptulose; and wherein said composition comprises amounts of the mannoheptulose sufficient to decrease a level of oxidized glutathione in the blood of the mammal subsequent to administration of the mannoheptulose.

27. The method of claim 26, wherein a ratio of reduced glutathione to oxidized glutathione in the blood subsequent to administration of the mannoheptulose is from about 0.1:1 to about 500:1.

28. The method of claim 26, wherein the level of oxidized glutathione in the blood subsequent to administration of the mannoheptulose is from about 0 μM to about 500 μM.

29. The method of claim 26, wherein said mannoheptulose is selected from the group consisting of glucose anti-metabolite, plant matter extract, avocado extract, and combinations thereof.

30. The method of claim 26, wherein the administration is oral.

31. The method of claim 26, wherein the composition comprises less than about 5% of said mannoheptulose, by weight of the composition.

32. The method of claim 26, wherein said composition is selected from the group consisting of pet food, dog food, cat food, treats, chew, biscuits, gravy, sauce, beverage, supplemental water, and combinations thereof.

33. The method of claim 26, wherein the composition is a nutritionally balanced pet food composition.

34. The method of claim 26, further comprising animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, lipids, and combinations thereof.

35. A method for decreasing inflammation and oxidative stress in a mammal comprising;
administration to a mammal a composition comprising avocado extract; and wherein said composition comprises amounts of the avocado extract sufficient to increase a ratio of reduced glutathione to oxidized glutathione in the blood of the mammal subsequent to administration of the avocado extract.
